(19) United States
(12) Patent Application Publication
(10) Pub. No.: US 2021/0231648 A1
(43) Pub. Date: Jul. 29, 2021

(54) LINKER MOLECULE FOR TREATING A SUBSTRATE SURFACE

(71) Applicant: SENZO HEALTH LIMITED, Altrincham (GB)

(72) Inventors: Aron RACHAMIM, London (GB); Ron OREN, London (GB)

(21) Appl. No.: 17/232,472

(22) Filed: Apr. 16, 2021

Related U.S. Application Data
(62) Division of application No. 15/745,380, filed on Jan. 16, 2018.
(60) Provisional application No. 62/194,186, filed on Jul. 17, 2015.

(51) Int. Cl. G01N 33/545; G01N 33/547; C07C 243/28; G01N 33/552; G01N 33/553

(52) U.S. Cl. CPC G01N 33/545; G01N 33/547; G01N 33/553; G01N 33/552; C07C 243/28

(57) ABSTRACT

A linker molecule and method for treating a substrate surface is provided, which includes a linker molecule with a plurality of moieties capable of resisting non-specific binding of proteins whilst permitting specific binding of a target biomolecule or a biomolecule of interest, including antibodies.

### RELATED APPLICATION

This application claims the benefit of U.S. Provisional Application No. 62/194,186 entitled “Linker Molecule for Treating Substrate Surface”, filed on 17 Jul. 2015, the subject matter of which is incorporated herein by reference in its entirety.

### CROSS REFERENCE TO RELATED APPLICATIONS

This application is a divisional of U.S. Ser. No. 15/745,380 filed Jan. 16, 2018 which is a National Phase of PCT Patent Application No. PCT/EP2016/066872 filed Jul. 15, 2016, which h claims the benefit of priority of U.S. Provisional Patent Application No. 62/194,186, filed Jul. 17, 2015, the contents of which are all incorporated herein by reference in their entirety.

### TECHNICAL FIELD

The present disclosure generally relates to linker molecules for treatment of a substrate surface used in detection of biomolecules. More particularly, this disclosure relates to a linker molecule with a plurality of moieties capable of resisting or suppressing non-specific binding of proteins and allowing specific binding of biomolecules including antibodies.

### DESCRIPTION OF RELATED ART

Medical devices including prosthetic devices, invasive devices and diagnostic testing kits, used for different medical purposes, are typically in contact with samples of bodily fluids such as blood or urine. Chemically, bodily fluids are highly complex with large amounts of proteins and salts dissolved, which can lead to significant problems due to deposition of the dissolved material on a solid support or a substrate surface. For example, deposition of large films of proteins and/or salts over a medical device can cause the device to malfunction. In another example, a thick film coating over a heart valve impairs its ability to flex thereby impairing fluid flow control. Similarly, a coating deposited on a sensor prevents other dissolved molecules from reaching the sensor, thus reducing the accuracy of the sensor.

Generally, the deposition of biomolecules from a solution onto a surface occurs via two pathways. First, a specific binding pathway may be used, where the surface is functionalized with active groups that bind to one specific type or family of biomolecules. Specific binding can be through biological interactions (e.g., antibody-antigen pairing or DNA hybridization) or chemical interactions (e.g., lock-and-key hydrogen bonding or ligand-metal pairing). Second, a non-specific binding pathway may be used, where biological molecules are deposited on the surface irrespective of the nature of these molecules. Non-specific binding can cause hydrophobic (van der Waals) interactions and electrostatic (charge-charge) interactions. The main challenge when designing medical devices is balancing between these two pathways. In many devices, specific binding is required for correct operation of the device, while non-specific binding reduces device efficiency. Therefore, there is a need to modify device surfaces such that they are protected from non-specific binding (“passivated”), while still allow specific binding of the target biomolecule (“activated”).

There exist references in the literature relating to the protein-resistant capacities of ethylene glycol based coatings. For example, Lee and Laibinis (Biomater., 1998, 19(18): 1669) reported oligo[ethylene glycol]-terminated alkyltrichlorosilanes that form 2-3 nm thick monolayers to provide near-perfect resistance to insulin, lysozyme, albumin, and hexokinase. US patent publication No. 2001/0031309 describes a silane molecule with a consecutive alkyl and oligo[ethylene glycol] chain. Oligo[ethylene glycol] chain with 4 ethylene glycol units, offers protection against non-specific binding of proteins. US patent publication No. 2009/0286435 describes a method of passivating a surface with n-substituted glyconic derivatives. The above literature teach that patterning of this glyconic derivative coating gives surfaces that both are protein resistant and protein binding, but this is only true on the macro-scale, whereas on the micro-scale (μm/mm), any area of the surface is either protein-resistant or protein-binding. Furthermore, the literature does nothing to actively promote protein binding to the surface, leaving protein binding to uncontrollable non-specific binding.

US patent publication No. 2009/0175765 describes a method to modify a glass or silicon surface with a mixed self-assembled monolayer, where one component is protein-resistant and the second component allows protein-binding. However, this method was shown only to work with ultra-low fractions of the second component, creating surfaces with one protein attached on every 7-10 μm², which is too little for surface-bound assays. US patent publication No. 2010/0041127 describes a method to coat a surface with a hydrogel carrying several bonding moieties. The hydrogel is functionalized with a protein resistant compound (methoxy-poly[ethyleneglycol]amine), and a target-binding ligand to activate the surface for selective target capture while resisting all other proteins. But, it is unclear how the fractions of protein-resistant and target-binding compounds on the hydrogel are controlled. Furthermore, hydrogels are known to be structurally flexible and capable of rearranging their surface in response to changes in the external medium; it is not confirmed that the hydrogel will continue to present the compounds in the targeted ratio on its surface when presented with a high ionic strength liquid (such as serum).

US patent publication No. 2005/0255514 describes a single molecule that combines protein resistance with activation for binding to specific biomolecules (such as DNA or proteins). The molecule has the generic form of A-(CH₂)n-(O[CH2CH2]x)m-(CH2)v-Y, where “A” is a silane-moiety capable of bonding to silicon, glass or similar surfaces; “Y” is a protein-binding moiety; —(OCH₂CH₂)m- provides protein-resistance and —(CH₂)n- is a non-active spacer. This disclosure acknowledges that the —(CH₂)n- spacer reduces protein-resistance and describes a secondary embodiment A-(CH₂)n-(OCH₂CH₂)m-Z, where “Z” is a hydrophilic capping group. European patent No. 0701697 takes a different approach towards depositing the passivating and binding agent, using an A-B-c block copolymer, where “A” is a hydrophobic block (poly[propylene oxide]) adhering to the surface; “B” is a protein-resistant block (poly[ethylene glycol]); and “c” is a reactive group allowing covalent connection to a protein of choice, wherein “c” may comprise a hydrazide (—NH—NH₂) group.

Thus, the prior art is limited to methods for coating a substrate surface with molecules conferring protein resistance and linker molecules allowing specific binding of biomolecules without providing protein resistance. Therefore, there still exists a gap in the art, which is addressed by the present disclosure.

It will be appreciated that reference herein to “preferred” or “preferably” is intended as exemplary only.

### BRIEF SUMMARY OF THE DISCLOSURE

The present disclosure relates to a linker molecule for treatment of a substrate surface, for providing biomolecule resistance (e.g., protein resistance) and allowing specific binding of biomolecules. In a broad form, the linker molecule prevents non-specific binding of proteins and allows specific binding of desired biomolecules (preferably a protein) or an analyte. The linker molecule comprises a hydroxyl binding moiety, capable of forming a covalent bond with activated hydroxyl groups on a substrate surface or a solid support. The linker molecule further comprises a biomolecule-resistant moiety having a segment of ethylene oxide or ethylene glycol with at least three repeating units. The linker molecule further comprises an antibody-binding moiety having a hydrazide group, capable of reacting with an aldehyde group on the antibody's Fc region of the antibody (also known as the stem region). Preferably, the biomolecule-resistant moiety is a protein resistant moiety.

The antibody-binding moiety is capable of binding to an antibody, without interfering with a biological function of the antibody. Preferably, the biological function of the antibody is the antigen-binding function. In an embodiment, the antibody binding moiety comprises a deprotected hydrazide group capable of reacting with an aldehyde group on Fc region or stem region of the antibody, after mild oxidation treatment. The Fc region is oxidized under mild oxidation conditions as is known in the art.

In an embodiment, the present disclosure relates to a method for depositing a linker molecule providing biomolecule resistance (e.g., protein resistance) and allowing specific binding of biomolecules to a substrate surface, the method comprising the steps of: i) covalently connecting the linker molecule to the substrate surface through 1-Ethyl-3-(3-dimethylaminopropyl) carbodiimide (EDC)-activated coupling; and ii) deprotecting a hydrazide group of the linker molecule, under mildly acidic conditions, for rendering the hydrazide group available for reaction with an antibody.

In another embodiment, the present disclosure relates to a synthetic route or synthetic pathway for producing a linker molecule that prevents non-specific binding of proteins and allows specific binding of desired biomolecules or an analyte. In another embodiment, a synthetic pathway for coupling an antibody to the linker molecule is disclosed. During synthesis of the linker molecule, the hydrazide group of antibody binding moiety is protected from side-reactions, for example, reactions with cleavable tBOC-group.

The articles “a” and “an” are used herein to refer to one or to more than one (i.e., to at least one) of the grammatical object of the article. By way of example, “an element” means one element or more than one element and should not be taken as meaning or defining “one” or a “single” element or feature. As used herein, the use of the singular includes the plural (and vice versa) unless specifically stated otherwise.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements. In some embodiments, the phrase “consisting essentially of” in the context of a recited subunit sequence indicates that the sequence may comprise at least one additional upstream subunit (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more upstream subunits) and/or at least one additional downstream subunit (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more upstream subunits), wherein the number of upstream subunits and the number of downstream subunits are independently selectable.

Additional objects, advantages, and novel features will be set forth in part in the detailed description, which follows, and in part will become apparent to those skilled in the art upon examination of the following detailed description and the accompanying drawings or may be learned by production or operation of the example embodiments. The objects and advantages of the concepts may be realized and attained by means of the methodologies, instrumentalities, and combinations particularly pointed out in the appended claims.

### BRIEF DESCRIPTION OF THE DRAWINGS

FIG. 1A shows a diagrammatic representation of the linker molecule, according to an embodiment of the present disclosure, which is not necessarily drawn to scale.

FIG. 1B shows an exemplary structure of the linker molecule comprising a plurality of moieties, according to an embodiment of the present disclosure.

FIG. 2 shows a synthetic route to an example linker molecule with shortest acceptable protein-resistant moiety, according to an embodiment of the present disclosure.

FIG. 3 illustrates an exemplary method of depositing a linker molecule onto a hydroxyl bearing support, followed by deprotection of the hydrazide moiety, according to an embodiment of the present disclosure.

### DETAILED DESCRIPTION

The following detailed description includes references to the accompanying drawings, which form a part of the detailed description. The drawings show illustrations in accordance with example embodiments. These example embodiments, which are also referred to herein as “examples,” are described in enough detail to enable those skilled in the art to practice the present subject matter.

The embodiments can be combined, other embodiments can be utilized, or structural, logical and operational changes can be made without departing from the scope of what is claimed. The following detailed description is, therefore, not to be taken in a limiting sense, and the scope is defined by the appended claims and their equivalents.

Surface treated substrates are useful for specific binding of chemical or biomolecules such as proteins (or fragments thereof), peptides, polypeptides, nucleotides, polynucleotides, small molecules, small organic molecules, biotin, cells, fractionated cells, cells extracts, cell fractions, parts of cells and other chemical or biological molecules that are of interest in the areas of, for example, proteomics, genomics, pharmaceuticals, drug discovery, and diagnostic studies.

As used herein, the term “substrate” refers to a solid support or a medical device surface or an inorganic or an organic substrate material. Substrates may comprise, but are not limited to, hard engineered surfaces such as silicon, glass, silica, quartz, metal oxides, indium tin oxide (ITO), mica, and the like. Organic substrates may comprise but are not limited to oxidized polymeric surfaces such as polyvinyl alcohol polymers, acrylic acid polymer, poly(methyl methacrylate) (PMMA), polystyrene, polycarbonate, polyvinyl chloride (PVC), and selected large molecules such as dissolved hydroxyl-bearing polymers (e.g., poly(2-hydroxyethyl methacrylate) (PHEMA), or P[OEGMA-OH]) or hydroxyl-bearing proteins. The substrates can be in the form of an optical fibre, wire, wafer, discs, planar surfaces, microscope slides, or beads. The substrate can also be a sensor, a biosensor, a DNA chip, a protein chip, a microarray, a microscope slide, a silicon wafer, or a microelectronic surface.

As used herein, the terms “target molecule,” “chemical or biological molecules,” “biomolecules,” and “desired biomolecules” refer to any specific binding substances that can be attached to the functionalized substrate surface or substrate surface.

As used herein, the term “aldehyde” refers to the molecules having the formula —CHO. In particular, the aldehyde groups found in the Fc region of an antibody are involved in coupling to an antibody binding moiety of a linker molecule.

As used herein, the term “surface” refers to any solid support surface that is capable of binding specific binding substances, either directly or indirectly.

The term “protein,” as used herein, means any protein, including, but not limited to peptides, enzymes, glycoproteins, protein hormones, receptors, antigens, antibodies, growth factors, and so forth.

Chemical or biological molecules can be selected from a group consisting of, for example, proteins, peptides, polypeptides, nucleotides, polynucleotides, small molecules, biotin, cells, fractionated cells, cells extracts, cell fractions, and parts of cells, and any combinations thereof.

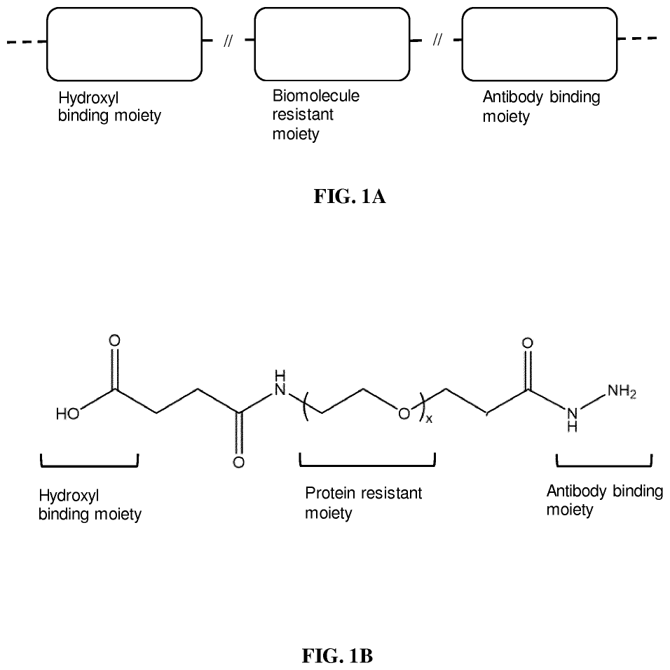

The present technology relates to a linker molecule for the treatment of a substrate surface. The linker molecule is configured to provide biomolecule resistance (e.g., protein resistance) and specific binding of biomolecules. In particular, the linker molecule is capable of resisting non-specific binding of a biomolecule (preferably, the biomolecule is a protein) and allowing specific binding of a target biomolecule or biomolecule of interest. Preferably, the target biomolecule or the biomolecule of interest a protein and more preferably, an antibody. In an embodiment, the linker molecule comprises a plurality of moieties, including: i) a hydroxyl binding moiety, which is capable of forming a covalent bond with activated hydroxyl groups on a substrate surface or a solid support; ii) a protein-resistant moiety comprising a segment of ethylene oxide or ethylene glycol with at least 3 repeating units; and iii) an antibody binding moiety comprising a hydrazide group, wherein the hydrazide group in deprotected form is capable of reacting with an aldehyde group on the antibody's non-functional region, and in particular the Fc region, as shown in FIG. 1A and FIG. 1B. It is contemplated that in certain embodiments of the present technology, the linker molecule consists of, or consists essentially of, a hydroxyl binding moiety capable of forming a covalent bond with activated hydroxyl groups on a substrate surface; a protein-resistant moiety comprising a segment of ethylene oxide or ethylene glycol with at least three repeating units; and an antibody binding moiety comprising a hydrazide group and capable of reacting with aldehyde groups on an antibody's non-functional region. In an embodiment, the binding region comprises a Fc region of the antibody. Preferably the non-functional region is a region of the antibody that does not bind to or interact with an antigen. More preferably, the region that does not bind to or interact with an antigen is a stem region or Fc region of the antibody.

The hydroxyl binding moiety is configured to form a covalent bond with activated hydroxyl groups on free molecules or substrate surfaces. The substrate surface may comprise binding partners including hard-engineered surfaces, oxidized polymeric surfaces, nanoparticles, micro-particles, hydroxyl-bearing polymers or hydroxyl bearing proteins.

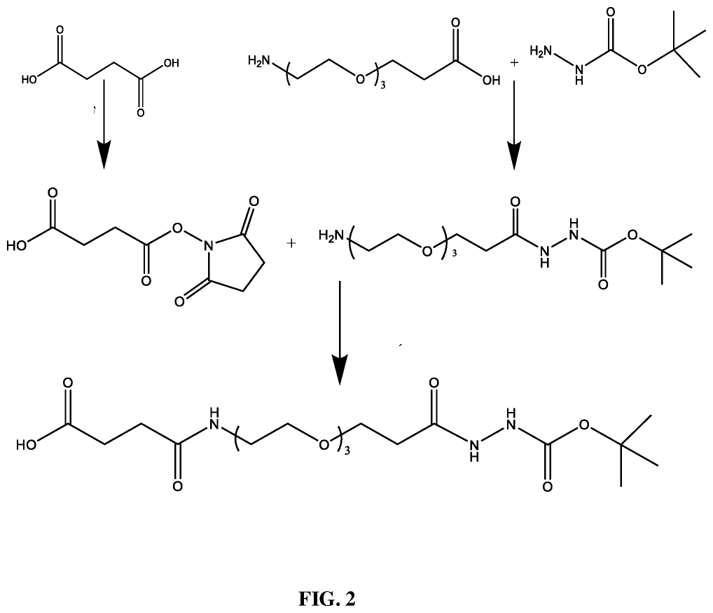

In an embodiment, the protein-resistant moiety is configured to block non-specific binding of proteins to the substrate surface. FIG. 2 shows a synthetic route for producing an example linker molecule with a shortest acceptable protein resistant moiety. The synthetic route is invariant to the length of protein-resistant moiety and needs only minor adjustment of the protocol, primarily in the purification steps to produce linker molecules with larger protein-resistant moieties. During the synthesis of the molecule, the highly reactive hydrazide group in the antibody-binding moiety is protected from side-reactions by the cleavable tBOC-group.

The linker molecule has been designed to offer simple deposition onto any substrate containing a hydroxy (—OH) group, including but not limited to oxidized silicon, oxidized glass, ITO, polymer substrates and free polymers. The linker molecule may comprise a linear molecule with at least three moieties namely a hydroxyl binding moiety, a biomolecule resistance moiety (and preferably, the biomolecule resistance moiety is a protein resistant moiety) and an antibody binding moiety as shown in FIGS. 1A and 1B.

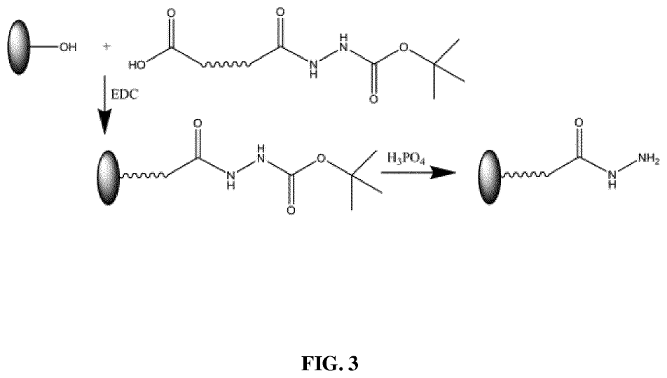

Referring to FIG. 3, which shows an exemplary method of depositing a linker molecule onto a hydroxyl bearing support, followed by deprotection of the hydrazide moiety, according to an embodiment of the present disclosure. The method comprises two separate steps: i) covalent connection of the linker molecule to the hydroxyl bearing substrate surface through EDC-activated coupling; and ii) deprotection of a hydrazide group under mildly acidic conditions, thus rendering the hydrazide group available for reaction with an antibody. Both of the above-mentioned steps in this reaction are compatible with organic and inorganic substrates, retaining structural properties and/or architectures of the modified substrate.

Once the linker molecule has been deposited onto the substrate surface and the hydrazide group is deprotected, it can be coupled to any antibody by interacting with the “stem” or “tail” of the antibody, such as the Fc region of the antibody. The antibody's Fc region is oxidized under mild oxidative conditions to allow this coupling, conditions for mild oxidation as would be appreciated by a person of ordinary skill in the art. Further, the Fc region of antibodies has been shown not to interfere with antibody functionality or antigen-antibody interaction. This need for oxidation of the antibody's Fc region provides an additional measure of control, by preventing unwanted bonding with a native antibody or antibodies normally found in a biological sample.

Generally, the Fc region shows low levels of variation between different antibodies. Thus, the linker molecule can be coupled with equal ease and efficiency to different types of antibodies. Furthermore, the Fc region does not participate in antigen binding so that coupling of antibody Fc region to the linker molecule and the substrate, does not affect a functional property, and in particular, the antigen-binding function of the antibody. In an embodiment, the deprotected hydrazide group is capable of reacting with an aldehyde group present in the Fc region of the antibody, after mild oxidation.

In an embodiment, the present disclosure relates to a linear molecule configured to provide resistance to or suppression of non-specific binding by proteins and allows specific binding to an antibody of interest. The linear molecule comprises a first active group configured to bind specifically to an antibody, a second active group configured to bind to a solid support or a substrate, and a third active group configured to block non-specific binding of proteins. The first active group and second active group are mutually compatible and orthogonal, allowing separate activation of the groups for a reaction. In an embodiment, each of the three active groups is protected from unwanted reactions or side reactions.

In another embodiment, the present disclosure relates to a synthetic route or synthetic pathway for producing a linker molecule that prevents non-specific binding of proteins and allows specific binding of desired biomolecules or an analyte. In another embodiment, a synthetic pathway for coupling an antibody to the linker molecule is disclosed.

Dissolved materials present in a biological sample, for example, proteins in a sample solution, tend to non-specifically bind to a substrate surface and interfere with the specific binding of desired biomolecules such as an antibody. For example, non-specific binding of proteins may occur via formation of van der Waals bonds and/or electrostatic interactions. On the other hand, specific binding can be through biological interactions (e.g., antibody-antigen pairing or DNA hybridization) or chemical interactions (e.g., lock-and-key hydrogen bonding or ligand-metal pairing). Therefore, the substrate surface needs to be treated in such a way to block non-specific binding of proteins and at the same time, to allow or promote specific binding of a target biomolecule or an analyte.

The surface of substrates needs to be protected from non-specific binding (passivated substrate) and should be allowing specific binding of the target molecule (activated substrate). The linker molecule of the present disclosure is configured to bind to any hydroxyl bearing substrate surface and further comprises active site for specific binding to a non-functional region of the antibody and a segment comprising repeated ethylene oxide or ethylene glycol units, which blocks non-specific binding of proteins.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms, without changing their ordinary meanings. The terms and expressions that have been employed are used as terms of description and not of limitation, and there is no intention that in the use of such terms and expressions of excluding any equivalents of the features shown and described or portions thereof, but it is recognized that various modifications are possible within the scope of the disclosure.

Although embodiments have been described with reference to specific example embodiments, it will be evident that various modifications and changes can be made to these example embodiments without departing from the broader spirit and scope of the present application. For example, such changes could include, but are not limited to, variations in the force used to actuate the valve membranes, variations in the rigid and soft materials, and variations and/or additions of further fluid or control layers. Accordingly, the specification and drawings are to be regarded in an illustrative rather than a restrictive sense.

1. A method for depositing a linker molecule providing biomolecule resistance and allowing binding of biomolecules to a substrate, the method comprising the steps of:
covalently connecting the linker molecule to the substrate through 1-Ethyl-3-(3 dimethylaminopropyl)carbodiimide (EDC) activated coupling; and
deprotecting a hydrazide group of the linker molecule, under mildly acidic conditions, for rendering the hydrazide group available for reaction with an antibody.

2. The method of claim 1, wherein the deprotected hydrazide group is configured to couple to a Fc region of the antibody.

3. The method of claim 1, wherein the linker molecule comprises a hydroxyl binding moiety, a protein binding moiety, and an antibody binding moiety.

4. The method of claim 1, wherein the substrate includes one or more of the following: silicon, glass, mica, ITO, an oxidized polymeric substrate, a free polymer, a nanoparticle, a microparticle, a hydroxyl-bearing support and a hydroxyl bearing polymer, and a hydroxyl bearing protein, and any combination thereof.

5. The method of claim 1, wherein the linker molecule is a linker molecule comprising:
a hydroxyl binding moiety configured to form a covalent bond with an activated hydroxyl group on a substrate surface through 1-Ethyl-3-(3 dimethylaminopropyl)carbodiimide (EDC) activated coupling;
a biomolecule-resistant moiety comprising a segment of ethylene oxide or ethylene glycol with at least three repeating units; and
an antibody binding moiety comprising a deprotected hydrazide group formed under mildly acidic conditions and configured to react with an aldehyde group on an oxidized Fc region of an antibody, wherein the F_{c} region is oxidized under mild oxidation conditions.

6. The method of claim 5, wherein the biomolecule-resistant moiety is a protein-resistant moiety.

7. The method of claim 5, wherein the hydroxyl binding moiety and the antibody binding moiety are mutually compatible and orthogonal.
